(19) United States
(12) Patent Application Publication
(10) Pub. No.: US 2009/0299369 A1
(43) Pub. Date: Dec. 3, 2009

(54) Hybrid Orthopedic Implant

(75) Inventors: Jorge L. Orbay, Miami, FL (US); Thomas H. Norman, Miami, FL (US); William Garcia de Quevedo, Miami, FL (US); Alejandro Espinosa, Miami, FL (US)

Correspondence Address: LERNER GREENBERG STEMER LLP, P O BOX 2480, HOLLYWOOD, FL, 33022-2480, US

(73) Assignee: SKELETAL DYNAMICS LLC, Miami, FL (US)

(21) Appl. No.: 12/476,408

(22) Filed: Jun. 2, 2009

Related U.S. Application Data
(60) Provisional application No. 61/058,046, filed on Jun. 2, 2008.

(51) Int. Cl. A61B 17/80

(52) U.S. Cl. 606/70; 606/286

(57) ABSTRACT

A hybrid orthopedic implant is provided. The implant includes a hybrid plate including a metal skeleton engaged with a plastic covering. Holes passing through the hybrid plate receive screws therethrough, to secure the hybrid plate to bone.

### CROSS-REFERENCE TO RELATED APPLICATION

The present application claims priority to the co-pending Provisional Patent Application No. 61/058,046, filed on Jun. 2, 2008 and entitled “Hybrid Orthopedic Implant”, which application is being incorporated herein, by reference, in its entirety.

### BACKGROUND OF THE INVENTION

1. Field of the Invention

The invention relates to a hybrid orthopedic implant.

2. Description of the Related Art

Orthopedic stabilization implants are commonly made out of metal. Plastic stabilization implants are used less frequently, as sufficient strength has generally not been available. Also, metal implants present the advantage of malleability; the surgeon can permanently change the shape of the implant to suit his needs by bending or twisting during application (intraoperatively). On the other hand, and because of their hardness, it is difficult for the surgeon to cut, or to shave, a metallic implant intraoperatively. Metal implants are normally manufactured by machining or forging the metal into the desired shape; therefore, it is costly to manufacture into complex or very thin shapes.

Plastic implants can be easily manufactured by molding, a process that permits easy forming into complex, thin shapes at low cost. Also, intraoperative size and shape modification is possible by means of cutting with scissors or shaving with a knife. Furthermore, plastic is more elastic and therefore will contour to the unique shape of a patient's bone, if made thin enough and pressed or molded onto the bone's surface. On the other hand, it is difficult to intraoperatively shape plastic implants by bending or twisting, because of their poor malleability.

A plate is a type of orthopedic stabilization implant that is applied to the surface of a bone in order to provide stability between two bone segments. Plates carry out their function by being securely attached to two bone segments by screws or by providing a buttressing effect to one of the bone segments while having screw attachment to the other. Frequently, stabilization plates have a head portion that is typically applied close to the metaphysis or end section of a bone and a shaft portion that is applied to diaphysis or middle section of bone. A neck portion, which connects these two parts, may also be present on the plate.

In certain situations, such as when correcting deformity, it is important that the neck portion be malleable in order to adjust its shape during surgery. This neck section is load-bearing, is usually away from anatomically sensitive areas and must be thick and strong, while remaining malleable. Metal has proven to be an optimal material for the neck and shaft sections of a plate.

The head portion of the plate is applied to the metaphysis and frequently provides a buttressing function. Here, the plate directly supports the surface of the bone and thus will contour optimally to its shape. Metaphyseal areas are always contiguous to joints, and tendons are usually in close proximity. For these reasons, it is preferable that this portion of the implant be as thin as possible in order to fit close to the bone surface and avoid tendon irritation. Because metal is difficult to manufacture into complex thin shapes and difficult to cut or shave in the operating room, it is often problematic to provide optimal buttress support with metal plates in those anatomically sensitive areas. Plastic has properties that are well suited for the metaphyseal portion of stabilization plates such as: a) plastic is easy to manufacture into a complex shape; b) plastic can be made into thin, elastic sections; c) plastic can be easily cut or shaved into the desired shape to fit the bone intraoperatively and d) plastic is a less irritating material to be in contact with moving tendons.

The screws that attach plates to bone are inserted through holes in the plate after drilling pilot holes into the bone. Often, it is desirable to insert these screws in directions that are not perpendicular to the central axis of the plate hole. Yet, frequently it is necessary that these screws lock in an angle-stable manner with the plate. Screws that self-tap into the plate provide an effective and simple method for obtaining this result. Because of its material properties, a plastic plate is well suited for providing this angle-stable engagement to metallic screws.

### BRIEF SUMMARY OF THE INVENTION

In order to overcome the above-mentioned disadvantages of the heretofore-known devices of this general type, it is accordingly an object of the invention to provide a hybrid orthopedic implant that is made of both metal and plastic and that derives the best properties from each material.

It is advantageous to have a metal skeleton or exoskeleton in the plate to provide optimal strength, load-bearing ability and the ability to be shaped by bending or twisting intraoperatively. The plastic covering the metal skeleton or attached to the metal exoskeleton allows the forming of complex shapes and thin sections to best adapt to and support the metaphysis while preventing tendon irritation. Self-tapping properties are provided by having screw holes in the metal skeleton or exoskeleton and the plastic covering.

Hybrid orthopedic implants made of plastic and metal present advantages by combining the benefits of each material and avoiding their disadvantages. The material that is strongest, has better deformation properties, or is easiest to manufacture or shape into complex or thin sections, can be selectively used for different portions of the implant.

Other features which are considered as characteristic for the invention are set forth in the appended claims.

Although the invention is illustrated and described herein as embodied in a hybrid orthopedic implant, it is nevertheless not intended to be limited to the details shown, since various modifications and structural changes may be made therein without departing from the spirit of the invention and within the scope and range of equivalents of the claims.

The construction of the invention, however, together with additional objects and advantages thereof will be best understood from the following description of the specific embodiment when read in connection with the accompanying drawings.

### BRIEF DESCRIPTION OF THE SEVERAL VIEWS OF THE DRAWING

FIG. 1 is a diagrammatic, top-plan view of a first embodiment of a hybrid plate according to the invention having a metal skeleton and a plastic layer;

FIG. 2 is an exploded, side-elevational view of the hybrid plate of FIG. 1;

FIG. 3 is an exploded, perspective view of the hybrid plate of FIG. 1;

FIG. 4 is a side-elevational view of an assembled hybrid plate of FIG. 1;

FIG. 5 is a perspective view of a second embodiment of a hybrid plate according to the invention having a metal mesh skeleton and a plastic covering;

FIG. 6 is a perspective view of a third embodiment of a hybrid plate according to the invention having a trabecular metal skeleton and a plastic covering;

FIG. 7 is a top-plan view of the hybrid plate of FIG. 6;

FIG. 8 is a cross-sectional view taken along the line A-A of FIG. 7, in the direction of the arrows;

FIG. 9 is a side-elevational view of the hybrid plate of FIG. 6; and

FIG. 10 is a perspective view of a fourth embodiment of a hybrid plate according to the invention having a metal skeleton and a plastic covering;

FIG. 11 is a perspective view of the metal skeleton portion of the hybrid plate of FIG. 10;

FIG. 12 is a top-plan view of the hybrid plate of FIG. 10

FIG. 13 is a cross-sectional view taken along the line B-B of FIG. 12 in the direction of the arrows.

FIG. 14 is a top-plan view of a hybrid plate, such as the hybrid plates of FIG. 5 or FIG. 6

FIG. 15 is a perspective view of the plate shown in FIG. 14

FIG. 16 is a cross sectional view taken along the line C-C of FIG. 14 in the direction of the arrows.

FIG. 17 is a side-elevational view of a fifth embodiment of a hybrid plate according to the invention having a metal exoskeleton and a plastic layer or covering

FIG. 18 is a top-plan view of the hybrid plate shown in FIG. 17; and

FIG. 19 is an end-elevational view of the hybrid plate shown in FIG. 18.

### DETAILED DESCRIPTION OF THE INVENTION

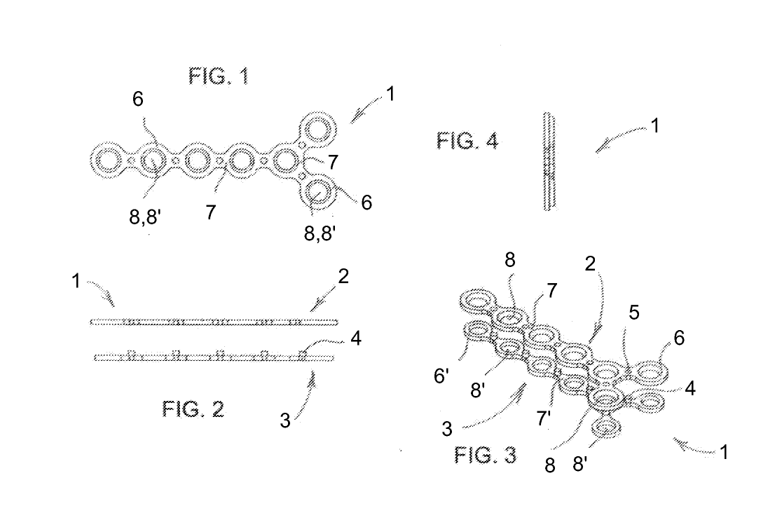

Referring now to the figures of the drawing in detail and first, particularly, to FIG. 1 thereof, there is seen a hybrid orthopedic plate 1 according to a first embodiment of the invention. It may be seen from FIGS. 2, 3 and 4 that the plate 1 has a body with a metal skeleton 2 and a plastic layer 3. Bosses 4 protruding from the plastic layer 3 are snapped or otherwise secured in corresponding holes 5 in the metal skeleton 2 in order to lock the elements 2, 3 together, as seen in FIGS. 1 and 4. The metal skeleton 2 has nodes 6, internodes or webs 7 between the nodes 6 and holes 8 passing through the nodes 6. The plastic layer 3 has nodes 6′, internodes or webs 7′ between the nodes 6′ and holes 8′ passing through the nodes 6′. Each pair of holes 8, 8′ receive one screw to be screwed into a bone and, preferably, self-tap in angle-stable position into one or both the metal skeleton 2 and the plastic layer 3 for holding the screws affixed to the plate and the plate affixed to the bone. The plate 1 may have any shape necessary for attachment to a bone or bones, such a linear shape, a curved shape, a Y-shape as shown, an L-shape, a polygonal shape, etc. Note that, if desired, in the present embodiment as well as in any of the embodiments that follow, the plastic layer 3 can be formed to include a peripheral edge or overhang that extends beyond the peripheral edge of the metal skeleton 2, thus permitting the size of the hybrid plate 1 to be adapted intraoperatively, i.e., through cutting or shaving of the overhang portion of the plastic layer 3. This permits the hybrid plate 1 to combine the malleability of metal with the sizeability of plastic. When making the hybrid plate 1, the amount of “overhang” provided in the plastic layer 3 can be chosen for, and/or adapted to, the particular application and/or anatomy to which the particular hybrid plate 1 is directed.

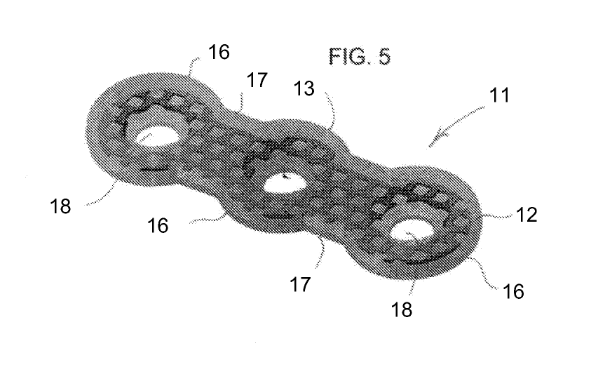

A second embodiment of a hybrid plate 11 is shown in FIG. 5. The plate has a body with a thin-walled metal mesh skeleton 12, for example, titanium, and a plastic layer 13, for example PEEK, covering the metal mesh skeleton 12. The plastic layer 13 may be flush with the metal mesh skeleton 12 or it may completely surround it. In a manner similar to the first embodiment, the hybrid plate 11 has nodes 16, internodes or webs 17 and holes 18 in the nodes for receiving screws. The plate 11 may have any required shape, as mentioned above. In the embodiment shown, the “mesh” body of the metal mesh skeleton 12 includes a plurality of holes or perforations therethrough, to better facilitate intraoperative bending of the hybrid plate 11. In particular, the perforations in the “mesh” of the metal mesh skeleton 12 are shown as being square in cross-section, although other cross-sectional shapes and/or amorphous cross-section can be used.

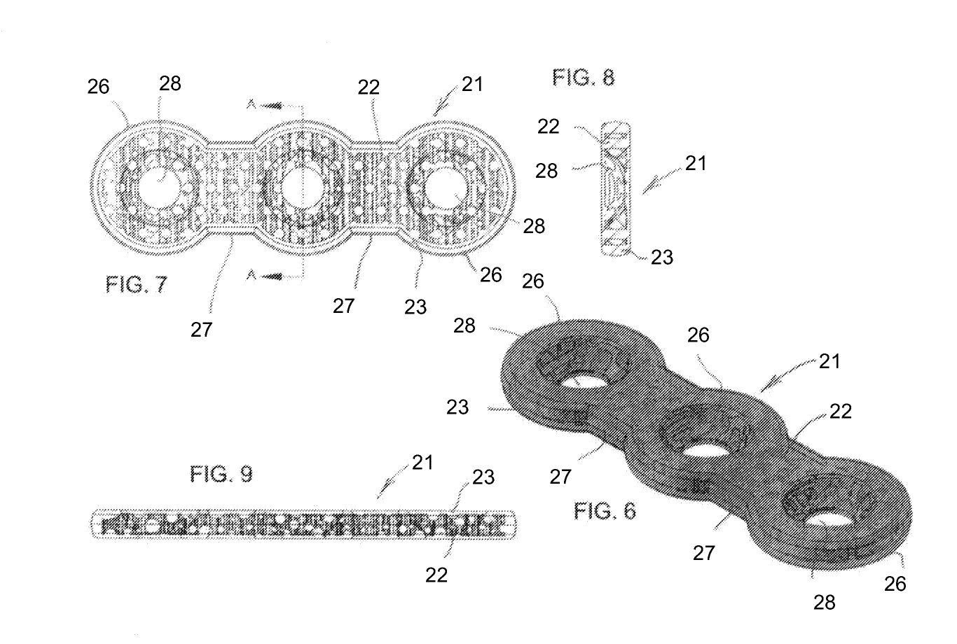

A third embodiment of a hybrid plate 21 is illustrated in FIGS. 6-9. The plate 21 has a body with a trabecular or foam metal core or skeleton 22, for instance titanium, and a plastic layer 23, for instance PEEK, covering the metal core 22. Once again, as in the first two embodiments, the hybrid plate 21 has nodes 26, internodes or webs 27 and holes 28 in the nodes for receiving screws. The hybrid plate 21 may have any of the shapes mentioned above and may additionally include perforations or holes through the core 22, to facilitate intraoperative bending of the plate 21. In the embodiment show in FIG. 7 the perforations are roughly circular in cross-section, although other cross-sectional shapes and/or amorphous cross-sections can be used.

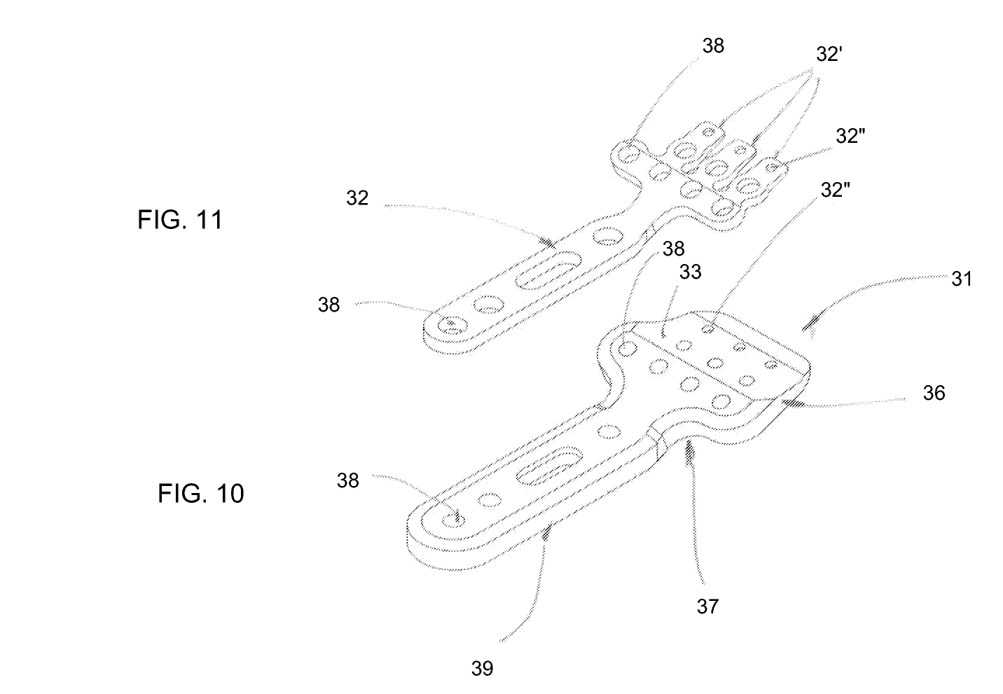

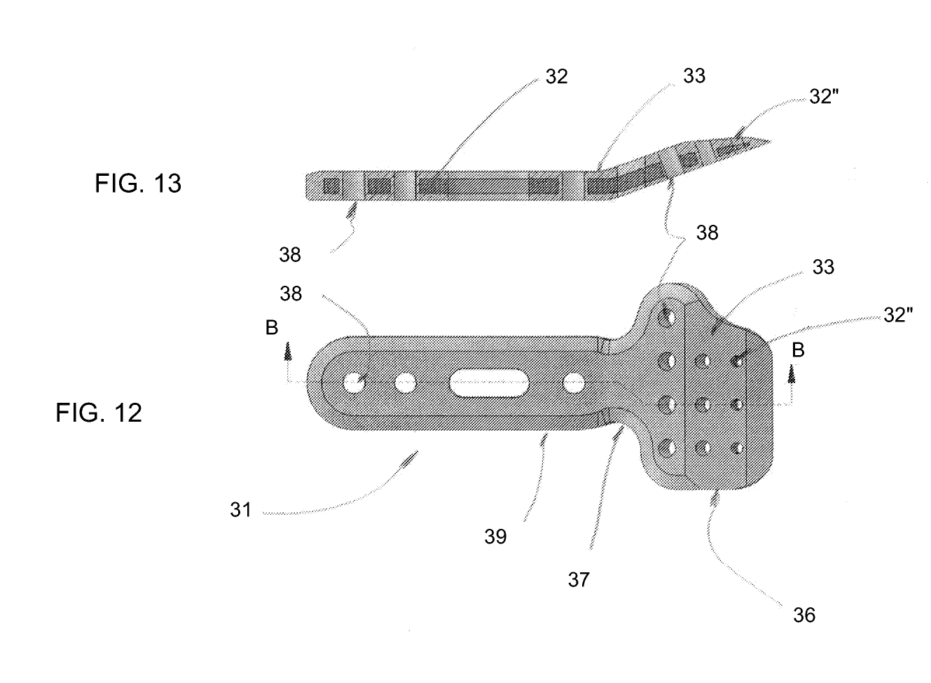

A fourth embodiment of a hybrid plate 31 is illustrated in FIGS. 10-13. The hybrid plate 31 has a body with a metal core or skeleton 32, for example titanium, and a plastic layer 33, made, for example of PEEK, covering the metal core 32. The hybrid plate 31 has a head portion 36, a neck portion 37, a shaft portion 39 and holes 38 in the head and shaft portion for receiving screws. The metal core or skeleton 32 may include tines 32′ at the distal edge of the head portion to facilitate differential bending or shaping of the head portion of the plate by engaging one or more bending tools into engagement holes 32″ and exercising torque.

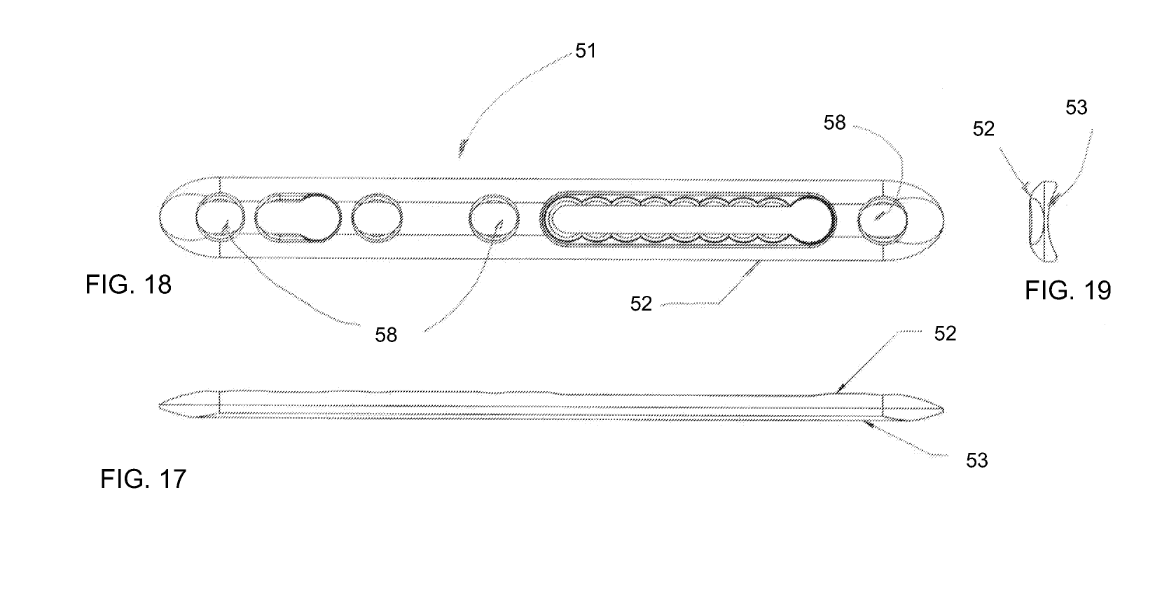

A fifth embodiment of a hybrid plate 51 is illustrated in FIGS. 17-19. The plate 51 has a body with a metal exoskeleton 52, for example, titanium, and a plastic layer or covering 53, for instance, PEEK, attached or fused to the metal exoskeleton 52. The hybrid plate 51 has holes 58 for receiving screws. The holes 58 can take any desired form, for example, circular, oval, keyhole and/or slotted, as shown in FIG. 18, without departing from the spirit of the instant invention. Further, a variety of types of screws, including, but not limited to, self-tapping screws, variable-angle screws and compression screws, may be used with the hybrid plate 51, or any of the other hybrid plates described herein, as desired.

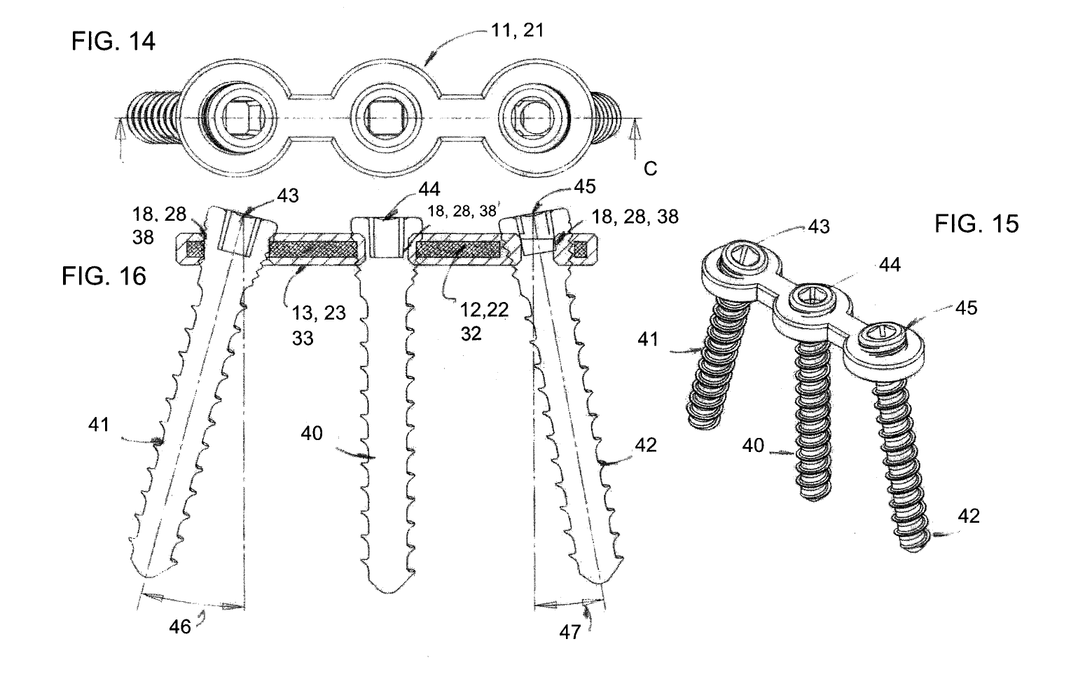

Referring now to FIGS. 14-16, there is shown a hybrid plate in accordance with certain embodiments of the present invention, for example, the hybrid plates 11 and 21, discussed in connection with the embodiments of FIGS. 5-9, herein. The plate 11, 21 has the metal or metal mesh core or skeleton 12, 22 and the plastic layer 13, 23 disposed thereon. Screws 40, 41, 42 pass through the holes 18, 28 and have self-tapping threaded portions 43, 44, 45 each retained in a respective hole in a node. Although the screw 40 is perpendicular to the plate, the screws 41 and 42 are disposed at angles 46 and 47 from the perpendicular in order to be screwed into a bone at an angle desired by the surgeon. Self-tapping portion 43 is shown tapping its own thread in angle-stable position into the metal core or skeleton 12, 22, 32 only; self-tapping portion 44 is shown tapping its own thread in angle-stable position into both, the metal core or skeleton 12, 22, 32 and the plastic layer 13, 23, 33. As can be seen more particularly in FIG. 16, the self-tapping portion 45 taps its own thread in an angle-stable position into the plastic layer 12, 23, 33, only. In a similar way, self-tapping portions 43, 44 and 45 of screws 41, 40 and 42 can self-tap threads in angle-stable positions into the metal core or exoskeleton, plastic layer or covering, or both, of holes 38, 58 of the fourth and fifth embodiments illustrated in FIGS. 10-13 and FIGS. 17-19, respectively.

1. A hybrid orthopedic implant, comprising:
a plate having nodes, internodes disposed between said nodes, and holes formed in said nodes;
said plate including a body with a metal core and a plastic layer disposed on said metal core; and
screws passing through said holes for attachment to a bone.

2. The hybrid orthopedic implant according to claim 1, wherein said metal core is a metal skeleton and said plastic layer is adjacent said metal skeleton.

3. The hybrid orthopedic implant according to claim 1, wherein said metal core is a metal mesh, and said plastic layer is PEEK at least partly surrounding said metal mesh.

4. The hybrid orthopedic implant according to claim 1, wherein said metal core is trabecular metal and said plastic layer is PEEK at least partly surrounding said trabecular metal.

5. The hybrid orthopedic implant according to claim 1, wherein said metal core is formed of titanium.

6. The hybrid orthopedic implant according to claim 1, wherein said screws pass through said holes for attachment to the bone and self-tap a thread into said metal core, said plastic layer or both in an angle-stable position selected by the surgeon intraoperatively.

7. A hybrid orthopedic implant, comprising:
a plate having a head portion, a shaft portion and a neck portion disposed between said head and shaft portions and holes formed in said head and shaft portions;
said plate including a body with a metal core and a plastic layer disposed on said metal core; and
screws passing through said holes for attachment to a bone.

8. The hybrid orthopedic implant according to claim 7, wherein said metal core is a metal skeleton and said plastic layer is adjacent to said metal skeleton.

9. The hybrid orthopedic implant according to claim 7, wherein said metal core is a metal mesh and said plastic layer is PEEK at least partly surrounding said metal core.

10. The hybrid orthopedic implant according to claim 7, wherein said metal core is trabecular metal and said plastic layer is PEEK at least partly surrounding said metal core.

11. The hybrid orthopedic implant according to claim 7, wherein said metal core is formed of titanium.

12. The hybrid orthopedic implant according to claim 7, wherein the distal portion of said metal core is divided into tines with engagement holes for accepting bending tools.

13. The hybrid orthopedic implant according to claim 7, wherein said screws pass through said holes for attachment to the bone and self-tap a thread into said metal core, said plastic layer or both in an angle-stable position selected by the surgeon intraoperatively.

14. A hybrid orthopedic implant, comprising:
a plate having a body comprising of a metal exoskeleton and a plastic layer or covering attached or fused to said metal exoskeleton and holes for receiving screws; and
screws passing through said holes for attachment to a bone.

15. The hybrid orthopedic implant according to claim 14, wherein said metal exoskeleton is a metal skeleton and said plastic layer is adjacent to said metal skeleton.

16. The hybrid orthopedic implant according to claim 14, wherein said screws pass through said holes for attachment to the bone and self-tap a thread into said metal exoskeleton, said plastic layer or covering, or both, in an angle-stable position selected by the surgeon intraoperatively.

17. A hybrid orthopedic plate, comprising:
a metal skeleton;
a plastic layer or covering attached or fused to said metal skeleton; and
at least said metal skeleton including holes for receiving screws through said holes for attachment to a bone.

18. The hybrid orthopedic implant according to claim 17, wherein said metal skeleton is malleable.

19. The hybrid orthopedic implant according to claim 18, wherein said metal skeleton is formed of a metal mesh.

20. The hybrid orthopedic implant according to claim 17, wherein said holes are configured to receive self-tapping screws therethrough to tap a thread into said metal skeleton, said plastic layer or covering, or both, in an angle-stable position selected by the surgeon intraoperatively.
